(19) United States
(12) Patent Application Publication
(10) Pub. No.: US 2025/0185935 A1
(43) Pub. Date: Jun. 12, 2025

(54) SYSTEMS AND METHODS FOR IN VIVO CHARACTERIZATION OF TUBULAR SOFT TISSUES

(71) Applicant: The General Hospital Corporation, Boston, MA (US)

(72) Inventors: Nima Saeidi, Brookline, MA (US); Dara Ahmadi Azar, Boston, MA (US); Shijie He, Brookline, MA (US)

(21) Appl. No.: 18/845,371

(22) PCT Filed: Mar. 8, 2023
(86) PCT No.: PCT/US2023/063958

§ 371 Date: Sep. 9, 2024

Related U.S. Application Data
(60) Provisional application No. 63/317,829, filed on Mar. 8, 2022.
(60) Provisional application No. 63/317,920, filed on Mar. 8, 2022.

(51) Int. Cl. A61B 5/03; A61B 1/273; A61B 5/00; A61M 25/10

(52) U.S. Cl. CPC A61B 5/036; A61B 1/2736; A61B 5/4238; A61B 5/6853; A61M 25/10182; A61M 2210/1053

(57) ABSTRACT

Systems and methods described herein can facilitate in vivo characterization of soft tissue, such as within a colon or other areas of a gut, by providing one or more of a geometric variable or a biomechanical response variable. For example, a tissue characterization system can include a control system, a pressure transducer, and a catheter. The system can measure pressure and diameter data of a soft tissue region or interest. The pressure and diameter data can be analyzed to provide a gut stiffness index that corresponds to various characterizations of the region of interest, such as an extent of gut fibrosis, a severity of mucosa damage, or an infiltration of immune cells

### CROSS-REFERENCE TO RELATED APPLICATIONS

This application claims priority to U.S. provisional patent application No. 63/317,920, filed Mar. 8, 2022, and U.S. provisional patent application No. 63/317,829, filed Mar. 8, 2022, each of which are entirely incorporated herein by reference.

### BACKGROUND

Gut stiffening caused by fibrosis is a hallmark of inflammatory bowel disease (IBD) and colon cancer. Previous studies have established that gut stiffening could lead to a manifestation of IBD and intestinal strictures. Moreover, stiffening is also reported to facilitate cancer metastasis.

Thus, it may be generally useful to characterize soft tissue regions within an anatomical space, such as the gut, to help determine a level of gut fibrosis, severity of mucosa damage to the gut, or infiltration of immune cells. However, the systems and methods for doing so are limited, at best.

### SUMMARY

Systems and methods described herein can facilitate in vivo characterization of soft tissue, such as within a colon or other areas of a gut, by providing one or more of a geometric variable or a biomechanical response variable.

In accordance with some non-limiting aspects of the disclosure, a system is provided for characterizing soft tissue within a soft tissue region of an anatomical body. The system can include a catheter configured to extend into the soft tissue region. A pump can actuate inflation or deflation of the catheter. A pressure transducer can be in communication with the pump. The pressure transducer can be configured to sense pressure within the catheter. A control system can be in communication with the pump to activate (e.g., inflate or deflate) the catheter within the soft tissue region and with the pressure transducer to record a series of pressure measurements from the catheter. The control system can be configured to coordinate pressure data from the catheter with diameter data from a series of diameter measurements of the soft tissue region. Additionally or alternatively, the control system can be configured to use at least one of the pressure measurements and the diameter measurements to determine a geometric variable or a biomechanical response variable that corresponds to a characteristic of the soft tissue.

In accordance with other aspects of the disclosure, a method for obtaining a gut stiffness index for a soft tissue region is provided. The method can include inserting a catheter into an anatomical space having a soft tissue region, inflating catheter within the anatomical space, inflating a soft tissue lumen within the soft tissue region by contacting the soft tissue lumen with the catheter, recording corresponding pressure and diameter data of the soft tissue lumen, calculating a stress versus strain curve from the pressure and diameter data of the soft tissue lumen, calculating the gut stiffness index from the slope of the stress versus strain curve, and generating a report including the gut stiffness index.

Other aspects of the disclosure can provide a method of characterizing soft tissue at a soft tissue region within a gut. The method can include collecting pressure measurements from the soft tissue region for a length of time to provide a first series of data points, collecting diameter measurements of the gut at the soft tissue region for the length of time to provide a second series of data points, calculating circumferential stress for each data point using the pressure measurements in the first series of data points and the corresponding diameter measurements in the second series of data points, calculating circumferential strain for each data point using the diameter measurements in the second series of data points and outputting one or more of a geometric variable or a biomechanical response variable that characterizes the soft tissue.

### BRIEF DESCRIPTION OF THE DRAWINGS

The patent or application file may contain at least one drawing executed in color. Copies of this patent or patent application publication with color drawing(s) will be provided by the Office upon request and payment of the necessary fee.

FIG. 1 is a schematic illustration of a soft tissue characterization system according to an example of the present disclosure.

FIG. 2 is a schematic illustration of a control system of the soft tissue characterization system of FIG. 1.

FIG. 3 is a schematic outline of software embedded in the control system of FIG. 2.

FIG. 4 illustrates hematoxylin and eosin stained ileum samples from a test site and an untested site of a test subject used in an exemplary experiment.

FIG. 5 includes graphical representation of data collected during the exemplary experiment; A) shows an inflation-deflation curve of a balloon catheter, B) shows a graph of gut pressure vs. diameter for in vivo and ex vivo tests, and C) shows a graph of gut pressure vs. diameter for various tissue regions.

FIG. 6 shows timelines of murine model development of the exemplary test; A) shows exemplary acute colitis development, and B) shows exemplary chronic colitis development.

FIG. 7 includes stress vs. strain graphs from the exemplary experiment; A) compares data from an acute colitis test with control data, and B) compares data from a chronic colitis test with control data.

FIG. 8 includes data collected in the exemplary experiment for a healthy group, an acute colitis group, and a chronic colitis group; A) shows a quantification of gut stiffness index, B) shows a quantification colon wall thickness, and C) shows a quantification colon outer diameter.

FIG. 9 shows various stains collected in the exemplary experiment for a healthy group, an acute colitis group, and a chronic colitis group; A) shows hematoxylin and eosin staining, B) shows a Masson's trichrome staining, and C) shows collagen IV immunofluorescent staining.

FIG. 10 includes metrics calculated in the exemplary experiment for a healthy group, an acute colitis group, and a chronic colitis group, the metrics include a fibrosis score (FS), a collagen-IV deposition intensity (CDI, and a gut stiffness index (GSI), A) shows a quantification of FS, B) shows a quantification of CDI, C) shows correlation coefficients for (GSI) vs. FS of acute colitis, D) shows correlation coefficients for GSI vs. CDI of acute colitis, E) shows correlation coefficients GSI vs. FS of chronic colitis, and F) shows correlation coefficients for GSI vs. CDI of chronic colitis.

FIG. 11 shows a stress-strain response for a proximal region and a distal region of an exemplary chronic colitis test colon used in the exemplary experiment.

FIG. 12 shows immunofluorescence staining of collagen-IV for the exemplary chronic test colon of FIG. 11, with more excessive collagen deposition and more severe damage of colon mucosa in the distal region.

FIG. 13 includes data collected for the exemplary chronic colitis test colon of FIG. 11 in the proximal region and the distal region, A) shows a quantification of gut stiffness index, B) shows a quantification of collagen deposition intensity, and C) shows correlation coefficients for gut stiffness index vs. collagen deposition intensity.

### DETAILED DESCRIPTION

The concepts disclosed in this discussion are described and illustrated with reference to exemplary arrangements. These concepts, however, are not limited in their application to the details of construction and the arrangement of components in the illustrative embodiments and are capable of being practiced or being carried out in various other ways. The terminology in this document is used for the purpose of description and should not be regarded as limiting. Words such as “including,” “comprising,” and “having” and variations thereof as used herein are meant to encompass the items listed thereafter, equivalents thereof, as well as additional items.

While the system and methods disclosed herein may be embodied in many different forms, several specific embodiments are discussed herein with the understanding that the embodiments described in the present disclosure are to be considered only exemplifications of the principles described herein, and the disclosed technology is not intended to be limited to the examples illustrated.

As briefly described above, gut stiffening caused by fibrosis can be an indication of inflammatory bowel disease (IBD) and colon cancer. Previous studies have established that gut stiffening could lead to the most severe manifestation of IBD and intestinal strictures. Moreover, stiffening is also reported to facilitate cancer metastasis.

Different strategies and devices have been used to measure gut stiffness, including in the large and small intestines, under pathological conditions. For instance, an indentation system and a measurement device (e.g., a device for measuring the modulus of stiffness of materials) have been used to determine the stiffness of excised healthy and fibrotic human gut samples ex vivo. Furthermore, conventional systems have utilized ultrasound elasticity imaging methods and can demonstrate an increase in the elastic modulus of fibrotic stenotic intestine in IBD patients.

While these conventional studies can demonstrate the translational value of using gut stiffness for the diagnosis of IBD, the methods of quantifying gut stiffness used in these studies are primarily conducive to ex vivo measurements, or lack adequate resolution, both of which limit their translational potential. In the present invention, we describe mechanoscopy for high resolution quantification of gut stiffness in vivo.

Mechanoscopy enables the acquisition of in vivo stress-strain curves of a gut, as we demonstrate, by way of example, in a colitis murine model. Ulcerative colitis (UC), as well as Crohn's disease (CD), is the common type of IBD. In one example described herein, based on a local slope of the stress-strain curves, we can define the gut stiffness index (GSI). The GSI can be efficiently applied to predict gut fibrosis and the severity of IBD. These results suggest mechanoscopy can be an avenue toward the translational application of gut stiffness measurement in IBD diagnosis.

Examples of the invention provide systems and methods for in vivo quantification of gut stiffness (i.e., mechanoscopy). A mechanoscopy system described herein can include a flexible balloon catheter, a pressure sensor, a syringe pump, and a control system. The control system can activate the balloon catheter and perform measurements (e.g., automated measurements) of a biomechanical response of a gut, such as a stress-strain biomechanical response. Further, the control system can generate a report related to gut pathology. For example, a gut stiffness index (GSI) can be identified based on the slope of the obtained stress-strain response. We have demonstrated that GSI positively correlates with the extent of gut fibrosis, the severity of mucosa damage, and the infiltration of immune cells. Furthermore, a critical strain value can be suggested and the GSI can efficiently detect pathological gut fibrotic stiffening when the strain exceeds this value. Based on these results, mechanoscopy and GSI can help facilitate clinical diagnosis of IBD, among other soft tissue characterizations, including detection of conditions with fibrosis in tubular tissue.

Described below are exemplary systems and methods, as well as testing of one example of a mechanoscope according to aspects of the present disclosure.

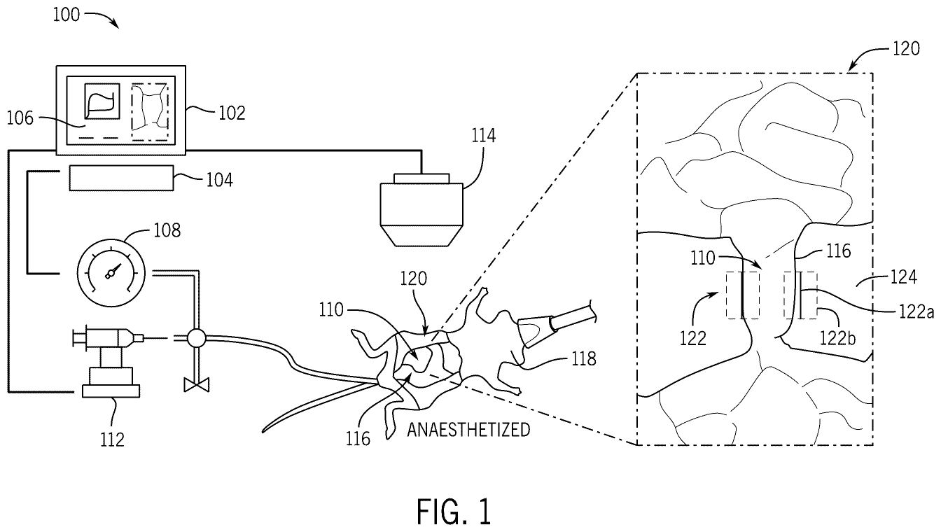

With reference to FIG. 1, a mechanoscope 100, according to one example, is shown. The mechanoscope 100 can be configured as a scope or gut measurement system configured to measure gut stiffness. In the present example, the mechanoscope 100 was built in a way which can be modified for either in vivo or ex vivo testing. However, it should be appreciated that systems and methods of soft tissue characterization described herein can be advantageously used in vivo, and that ex vivo testing capabilities may be optional or used to confirm results in test scenarios.

FIG. 1 shows the mechanoscope 100 being used in vivo. Under both in vivo and ex vivo modalities, the mechanoscope setup 100 can include the following components: a control system 102 (e.g., a LabVIEW-based control system) having a controller 104 and equipped with a gut diameter measurement module 106, a pressure transducer 108, a catheter 110 (which may be formed, in one example, as a water-filled balloon), and a syringe pump 112 for inflation/deflation of catheter 110. In some embodiments, the gut measurement system (e.g., the mechanoscope 100) can also include a camera 114 for diameter measurement. The control system 102 can further include or otherwise coordinate a real-time readout of diameter and pressure. For example, each instance the system 100 records measurements, the control system 102 can coordinate a diameter measurement with a pressure measurement for each instance of measurement acquisition.

For an in vivo mechanoscopy test, the deflated catheter 110 was situated inside a gut lumen 116 of a subject 118 (e.g., an anaesthetized mouse) via surgery. During the testing, mice were anesthetized via isoflurane as an anesthetic agent according to Institutional Animal Care and Use Committee (IACUC) guidelines. According to the example illustrated in FIG. 1, after laying down the mice 118 in a supine position, access to the abdomen 120 was provided via a U-shaped incision.

In the example test, a total of forty 25-30 g 10-week-old male C57BL/6J male mice were procured. The animals were housed and maintained on a 12-hour light/dark cycle with access to food and water ad libitum. All experimental procedures were approved by the Institutional Animal Care and Use Committee (IACUC) of the Massachusetts General Hospital and met the guidelines of National Institutes of Health (NIH). The mice were divided into 4 groups comprised of two disease models (acute colitis and chronic colitis) and a control group for each.

For in vivo studies of the jejunum and ileum, small vertical incisions were made for catheter placement 2 mm above the positions selected for ex vivo studies. For in vivo studies of the colon, the balloon catheter 110 was guided through the anus and placed at the required positions (proximal, mid, or distal). In the ex vivo setup, 12 mm specimens were dissected from the middle of the colon, proximal jejunum (30 mm after the ligament of Treitz), and distal ileum (5 mm above the cecum), respectively, and flushed with 37° C. phosphate-buffered saline (PBS). The dissected specimens were mounted in the bioreactor with a closed flow system based on oxygenated PBS to ensure intestinal tissue viability during testing.

The gut diameter measurement module 106 was embedded in the software of the control system 102 to ensure correct registration of pressure (P) and gut outer diameter (Dₒᵤₜₑᵣ) in each measurement. For a gut diameter measurement (see for example measurement lines 122 in FIG. 1), digital images (or video) of a gut at test sites were automatically sequenced and recorded via the camera 114. Any warp or distortion of the lens of the camera 114 was corrected via a standard dot grid calibration. Using a series of digital images acquired during test, the gut diameter measurement module 106 in the control system 102 can identify the gut 122a and detect the gut edges 122b to automatically calculate the gut outer diameter (Dₒᵤₜₑᵣ).

To facilitate gut diameter measurement, a black cloth 124 was placed under the section of interest to create the highest contrast background, as shown in FIG. 1. However, other high-contrast colored materials may be used. Using the control system 102, the balloon 110 was inflated at a rate of 0.5 ml/min under quasi-static loading while actively measuring the gut outer diameter and balloon pressure. The balloon pressure was recorded at a frequency of 1.9 Hz. During the inflation phase, the catheter 110 contacted the gut lumen 116 and inflated the gut to a predefined maximum radial stretch. We estimated (e.g., calculated) the maximum physiological circumferential stretch of the gut by measuring the changes in gut diameter during chyme digestion (e.g., ratios of stretched to original diameters include: 2±0.25, 2.2±0.16 and 2.1±0.15 for jejunum, ileum and colon, respectively) to ensure that the imposed radial stretch remained within physiological range. Initial gut diameter data was acquired from the average of several real-time camera detections before balloon inflation.

For ex vivo analysis, the sample was housed in a bioreactor equipped with a phosphate-buffered saline (PBS)-based perfusion/bathing system and a temperature sensor. The ex vivo balloon inflation/deflation measurement was identical to the above but performed in the bioreactor with perfused PBS at 37° C. With both the ex vivo and in vivo measurement, 5 cycles of inflation-deflation preconditioning up to 60% of the maximum radial strain were performed to minimize the viscous response and variability of the soft tissue. Following the preconditioning cycles, 3 inflation-deflation cycles were completed and collected during each test for further analysis. After the in vivo tests, we repeated the measurements with the ex vivo setup and confirmed that the measurements from the in vivo setup and the ex vivo setup were in accordance with each other. The animals fully recovered after mechanoscopy and behaved normally.

As described above, the mechanoscope 100 for gut stiffness measurement, according to examples of the present disclosure, can include the control system 102, the pressure transducer 108, the catheter 110, the syringe 112 for catheter 110 inflation/deflation, the diameter-detecting camera 114, and gut diameter measurement module 106 (see, for example, FIG. 1). By way of example, the control system 102 can include a LabVIEW module. The catheter 110, which may be generally configured as a balloon probe that can characterize tissue and link acute material response to an existence of a soft tissue disease and provide an indication of the severity of the disease.

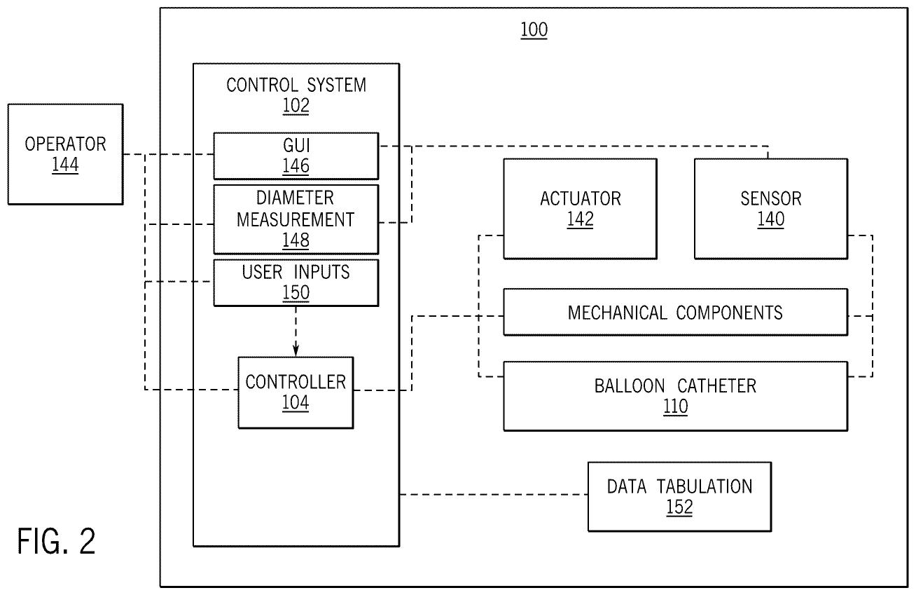

With reference to FIG. 2, the control system 102 can control a variety of functions via the controller 104, including: (1) initiate communication with various system components (e.g., sensors 140 and actuators 142), (2) actuate mechanical components (e.g., balloon 110 inflation/deflation), (3) interact with an operator 144 via a graphic interface (e.g., GUI) 146, real-time visualization of diameter measurement 148, user inputs 150 and errors, (4) automate repeatable tasks, and (5) tabulate data 152 for further analysis. In some examples, the pressure and gut diameter can be measured in real-time at a frequency of 1.9 Hz (114 times per minute). In other examples, the pressure can be measured at other frequencies, such as between 1.4 Hz and 3 Hz

When the mechanoscope system 100 is in use, images (or video) can be acquired using a scope to estimate lumen geometry at every balloon 110 position within the region of interest. Pressure data from the balloon 110 can be recorded using a sensor that measures fluid pressure inside the balloon probe 110. By way of example, the balloon catheter or probe 110 does not need to rely on impedance sensing.

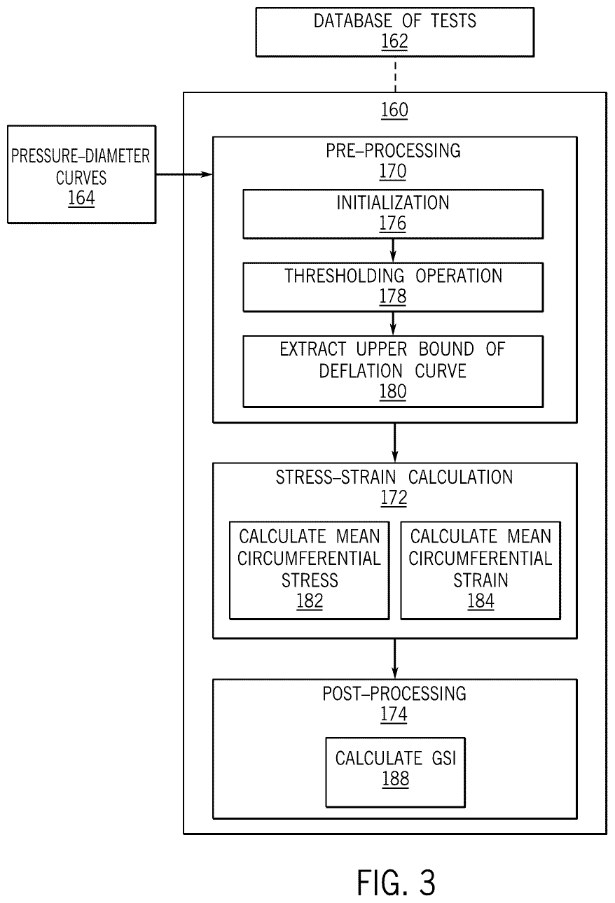

With reference now to FIG. 3, and in accordance with examples of the present invention, we developed mechanoscopy software 160. The mechanoscopy software 160 is based on a database of tests 162 (e.g., 480 tests) to automatically analyze obtained pressure-diameter curves 164 (e.g., inflation or deflation curves). The software algorithm consists of (1) preprocessing 170, (2) stress-strain calculation 172, and (3) postprocessing 174. In the preprocessing phase 170, following an initialization 176, any unrealistic data entry caused by sensor noise is removed via a thresholding operation 178. The upper bound of the deflation curves 164 can be extracted at step 180 by detecting the farthest point. In the software, this datapoint is identified as the one at the greatest Euclidean distance from the origin (0,0) in the total pressure (Pₜₒₜₐₗ)−outer diameter (Dₒᵤₜₑᵣ) plot.

With reference again to the exemplary testing, the gut pressure P_{gut} was calculated by

$${P}_{gut} = {P}_{total} - Z \qquad ( Equation 1 )$$

where Z is the residual pressure of the balloon catheter calculated based on the initial intersecting outer diameter and a linear fit of the plateau region of the deflation curve. To ensure systematic averaging across multiple iterations, gut pressure P_{gut} values are defined based on groupings within a width of 5 mmHg. At steps 182 and 184 of the stress-strain calculation phase 172, mean circumferential stress and mean circumferential strain are then calculated for each measured pressure-diameter data point as

$$Circumferential Stress σ = \frac{{P}_{gut} {D}_{inner}}{{D}_{outer} - {D}_{inner}} \qquad ( Equation 2 )$$

$$Circumferential Strain ε = \frac{{D}_{inner}}{{D}_{outer , 0} - 2 t} \qquad ( Equation 3 )$$

where D_{outer,0} and t are, respectively, the initial undeformed diameter and the undeformed wall thickness. Dᵢₙₙₑᵣ is the deformed inner diameter calculated based on the assumption of incompressibility

$${D}_{inner} = 2 \times \sqrt{{( \frac{{D}_{outer}}{2} )}^{2} - {( \frac{{D}_{outer , 0}}{2} )}^{2} + {( \frac{{D}_{outer , 0}}{2} - t )}^{2}} \qquad ( Equation 4 )$$

To quantify the gut biomechanical response from the postprocessing results, the local slope of the stress-strain curve was defined as the gut stiffness index (GSI). Interpolation between measured data points was performed using the Gaussian Kernel Regression model. Then, using a Linear Regressor in the neighborhood of a given strain, the GSI was calculated, as shown in step 188 of FIG. 3. The neighborhood radius is a hyper-parameter that can be modified by the user, and, here, is set to 1, however, other values are possible. In some examples, the GSI can be outputted via a report comprehendible to a user or clinician.

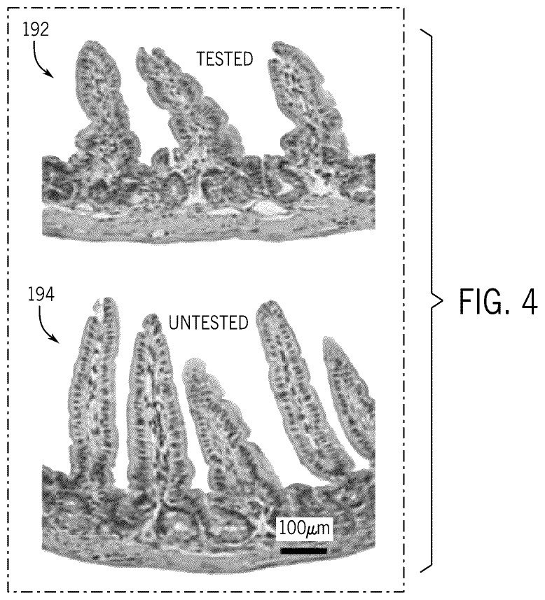

In one example, to verify the safety of the gut stiffness measurement using mechanoscope 100, after the measurement, ileum samples from the test sites and the sites adjacent to them were collected for hematoxylin and eosin (H&E) staining (see, for example, FIG. 4). No damage was detected in the samples from the test sites (e.g., a mucosa and muscle layer), as shown by the comparison of the H&E stain for the tested sample 192 and the untested sample 194 shown in FIG. 4. The intact crypt-villus architecture and the continuous structure of the submucosa and muscle were maintained, as shown in FIG. 4.

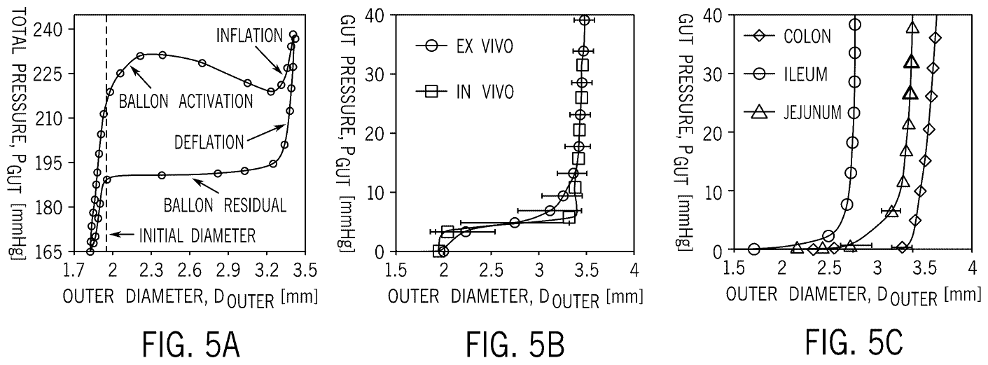

In testing cycles, the gut outer diameter Dₒᵤₜₑᵣ was recorded in correspondence with the total pressure Pₜₒₜₐₗ in the balloon probe. Four phases were included in each testing cycle: balloon activation, balloon inflation, balloon deflation, and balloon residual. FIG. 5A shows a representative inflation-deflation curve based on Pₜₒₜₐₗ and Dₒᵤₜₑᵣ at a steady state. The vertical dashed line on the left side of the graph marks the gut diameter at rest prior to testing.

The gut pressure P_{gut} was calculated by subtracting the balloon residual from Pₜₒₜₐₗ. In each test, the gut pressure P_{gut} and the corresponding outer diameter Dₒᵤₜₑᵣ from the deflation curve were used for the gut stiffness measurement. We performed the mechanoscope ex vivo and in vivo for ileum samples from 20-week-old healthy mice (see FIG. 5B). The P_{gut}−Dₒᵤₜₑᵣ curves were acquired following five systematic preconditioning cycles. The in vivo and ex vivo results were in accordance with one another, which confirmed the repeatability and accuracy of the in vivo measurement using mechanoscope 100. The in vivo setup was leveraged to record gut biomechanical response for the remaining experiments, which were then verified with the ex vivo setup.

Jejunum, ileum, and colon were used to examine whether the mechanoscope is capable of producing gut segment-specific biomechanical response. Each segment exhibited a distinct pressure-diameter curve when P_{gut} changed from 0 to 40 mmHg (see FIG. 5C).

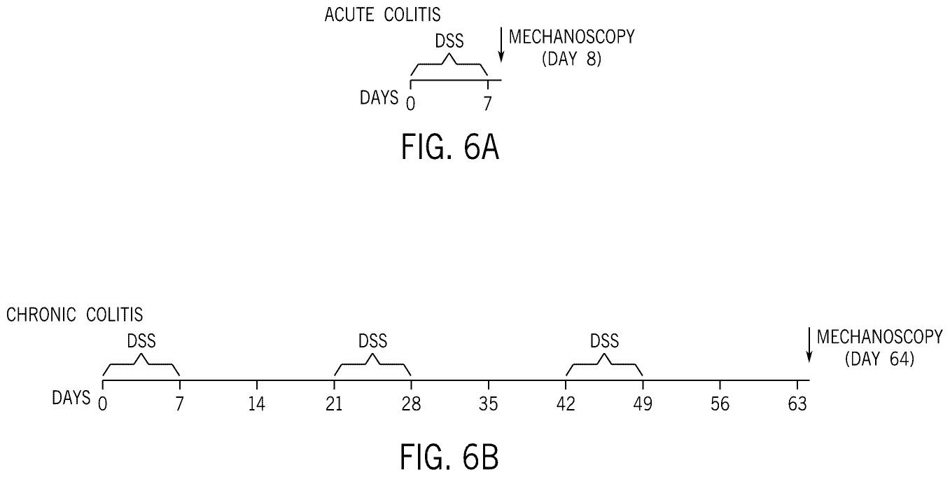

With reference to FIG. 6, the colitis murine model was developed by introducing dextran sulfate sodium (DSS) to drinking water over a controlled regimen and duration. Acute colitis was developed by administering 3% (wt/vol) DSS (molecular weight 36-50 kDa, MP Biomedicals, Irvine, CA) in drinking water for 7 consecutive days (see FIG. 6A). To induce chronic colitis, DSS was administered 1 week followed by normal water for 2 weeks for a total of 3 cycles (see FIG. 6B). Control groups included age- and sex-matched animals that were only given normal water. Animals were observed daily for body weight, water/food consumption, stool consistency, and presence of blood in feces and around the anus. The animals showed signs of inflammation (e.g., body weight loss, blood in stools, and piloerection) approximately five days after initiation of DSS administration which fully resolved by the end of the first recovery week.

After the mechanoscope measurement, tissue samples from the jejunum, ileum, and colon collected from the tested/untested site were fixed in 10% formalin and further processed for histological studies. Following sectioning at 5-μm thickness, prepared sections were deparaffinized and then stained with hematoxylin and eosin (H&E), Masson's trichrome, and the DAPI-TRITC combination for collagen IV staining. This can be used to calculate a fibrosis score and immunofluorescent intensity. In general, colon tissue was the most affected tissue during DSS-induced colitis, and colon biomechanical properties were measured using the mechanoscope 100.

A correlation coefficient was calculated based on the correlation function,

$$Corre ( X , Y ) = \frac{\sum ( x - \overline{x} ) ( y - \overline{y} )}{\sum {( x - \overline{x} )}^{2} {( y - \overline{y} )}^{2}} \qquad ( Equation 5 )$$

where x̅ and y̅ are, respectively, the average of the data X and data Y.

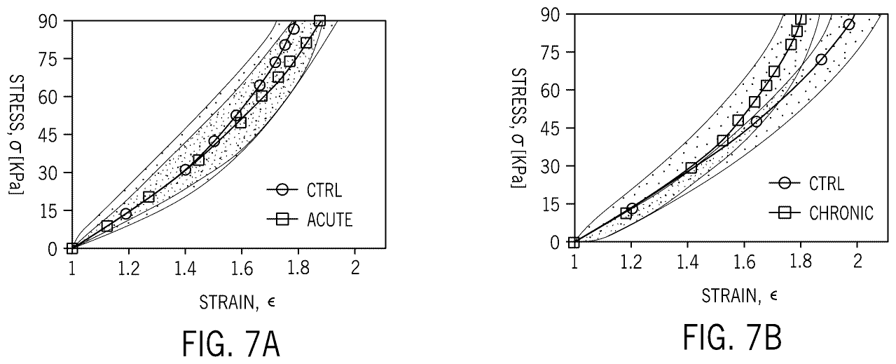

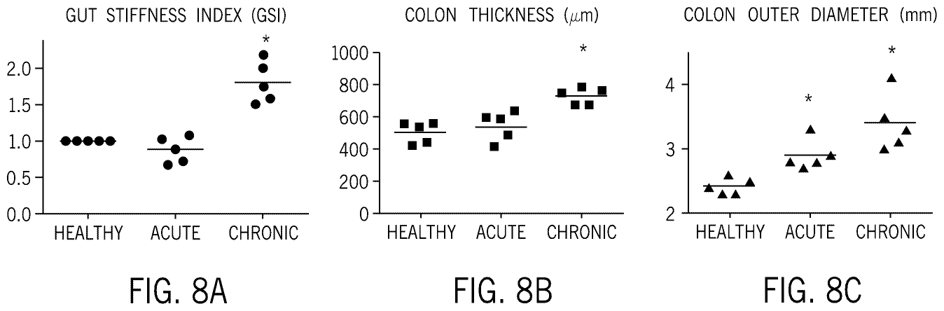

In the case of DSS-induced acute colitis, the stress-strain responses (Equation 2 and Equation 3) nearly overlapped with the healthy control group, suggesting there was no significant difference between the healthy group and the acute colitis group in terms of gut stiffness (see FIG. 7A). However, in the case of chronic colitis, at strains ε>1.5, the stress-strain response exhibited a more severe stiffening property (more abrupt slope of the stress-strain curve) compared to the healthy group (see FIG. 7B). In each of the graphs of FIGS. 7A and 7B, the gray area around the lines corresponds to standard deviation. This result provided a critical divergence point for distinguishing the stress-strain response in chronic colitis which is at ε=1.5. Beyond this point the colon of the chronic colitis group became more stiffened than the healthy group. Thus, reaching the critical strain level is required for detecting the divergent biomechanical response in chronic colitis. The local slope of the stress-strain curves was used to define the gut stiffness index, GSI. Compared to the healthy control, GSI measured at ε=1.6 showed a non-significant change for the acute group, but significantly increased about 1.8 times for the chronic group (FIG. 8A).

In addition, compared to the healthy group, the wall thickness of the chronic colitis colon increased by 42% (see FIG. 8B). The initial outer diameter D_{outer,0} of the colon increased by 8.6% in the acute colitis group whereas it increased by 19.3% in the chronic colitis group (see FIG. 8C). The thickening of the colon wall and the greater increase of the colon outer diameter observed in chronic colitis were accompanied by colon stiffening (see FIGS. 8A-C).

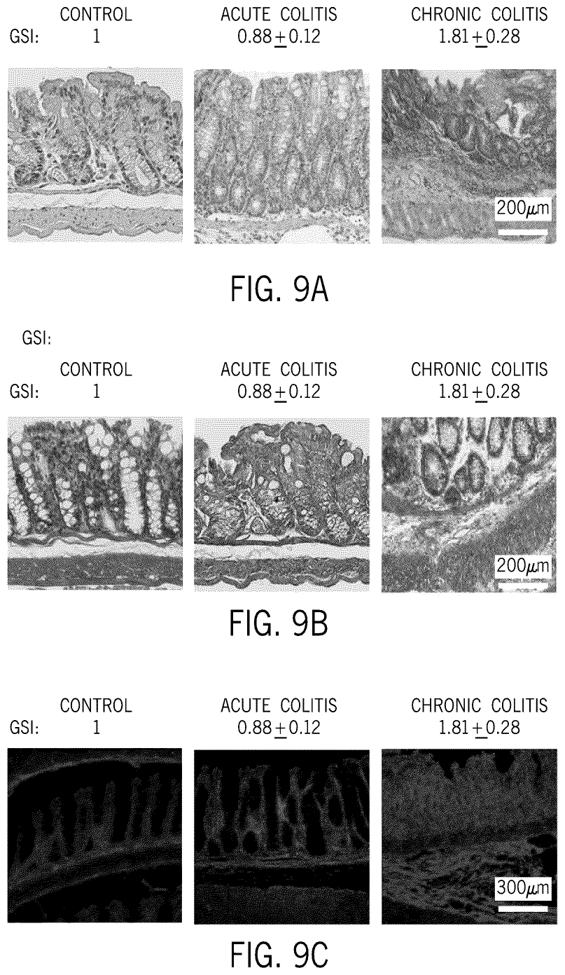

With reference to FIGS. 9A-C, colon GSI was positively correlated tissue fibrosis and severity of the DSS-induced colitis. In both acute and chronic models, disease induction was characterized by body weight loss, appearance of diarrhea, and visible fecal blood. The most recurring and severe symptoms were observed in the chronic model during the last DSS feeding cycle. H&E staining showed that compared to the healthy and acute colitis groups, the damage of colon mucosa and immune cell infiltration were most severe in the chronic groups (see, for example, FIG. 9A). Moreover, as a sign of fibrosis, collagen deposition was greater for the chronic colitis group compared to the acute colitis group as shown in both Masson's Trichrome staining in FIG. 9B and collagen-IV immunofluorescent staining shown in FIG. 9C.

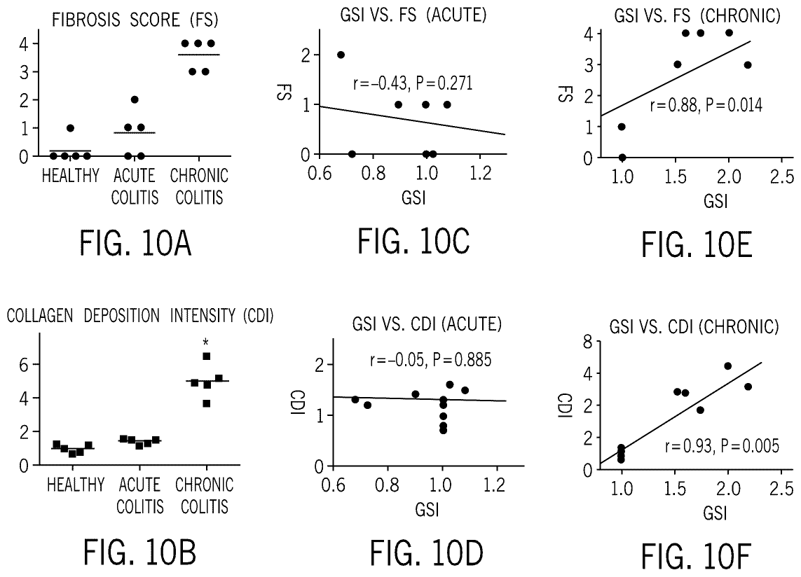

Based on the Masson's Trichrome staining (e.g., FIG. 9B) and the collagen immunofluorescent staining (e.g., FIG. 9C), we calculated the fibrosis score (FS), shown in FIG. 10A, the normalized collagen deposition intensity (CDI), shown in FIG. 10B, and their correlation coefficient with GSI. For FS, ‘0’ is scored for no fibrosis and the higher the score, the more severe the fibrosis. While there were no significant differences between healthy colon and acute colitis colon with respect to the FS and CDI, they were both significantly greater in the chronic colitis colon. Moreover, in the acute colitis colon, the correlation coefficients of the FS vs. GSI and CDI vs. GSI were −0.43 and −0.05, respectively, suggesting an absence of any correlations between the GSI and fibrosis severity (see FIGS. 10C and 10D). For the chronic colitis colon, the correlation coefficients were, 0.87 for FS vs. GSI, shown in FIG. 10E and 0.92 for CDI vs. GSI, shown in FIG. 10F, indicating that the GSI score was strongly and positively correlated with fibrosis severity in the chronic colitis group. Therefore, the quantification of colon biomechanical score GSI using the mechanoscope 100 can be used for the diagnosis of gut fibrosis and determining the severity of the pathological damage induced by IBD.

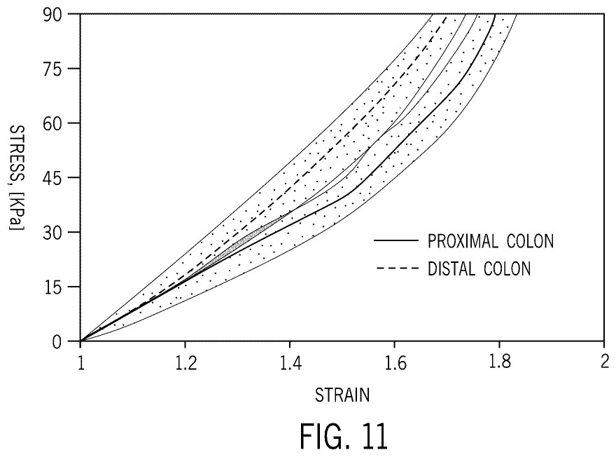

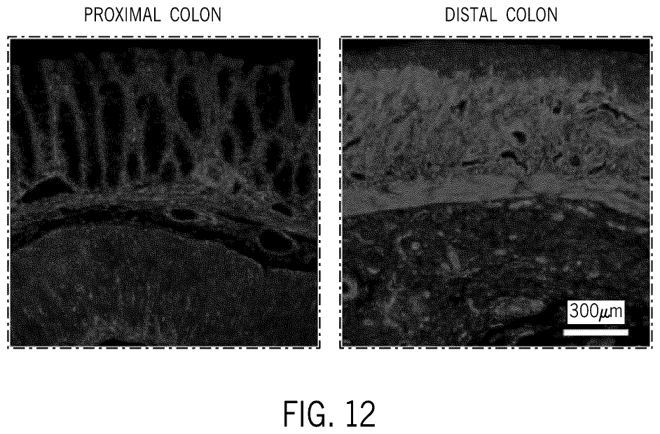

In other examples, the mechanoscope system 100 can be employed at various soft tissue sites. In one example, using the mechanoscope 100, we diagnosed the non-uniform region-dependent development of chronic colitis in mouse colon. The biomechanical response of the proximal colon and the distal colon were measured (see FIG. 11), and after the measurement, the tissues from the same corresponding sites were collected for histology analysis (see FIG. 12). The in situ in vivo stress-strain responses showed that compared to the proximal colon in the same mice with chronic colitis, stiffening (slope of the stress-strain curves) was more severe in the distal colon, as highlighted in FIG. 11. GSI at ε=1.6 increased by 19% in the distal colon compared to the proximal colon (see FIG. 12).

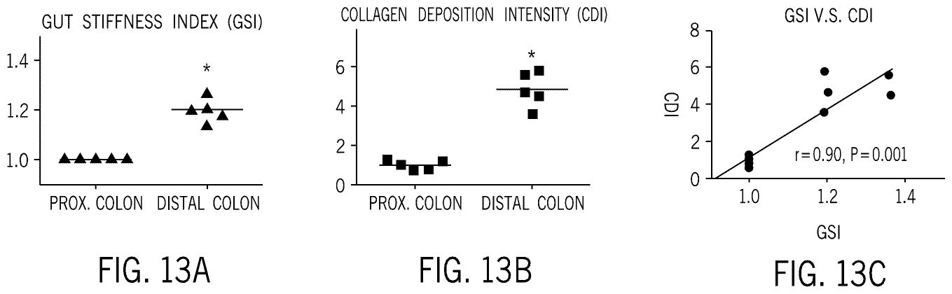

Correspondingly, compared to the proximal colon, the collagen deposition and the mucosa damage in the distal colon were more severe (see, for example. FIGS. 13A and 13B). The correlation coefficient of CDI vs. GSI across the proximal and distal colon is 0.90 (see, for example, FIG. 13C), showing that the increase of GSI in the distal colon is strongly and positively correlated with the increase of fibrosis severity in the distal colon.

Unlike conventional methods characterizing biomechanical response of gut in vitro or ex vivo, embodiments described herein provide a novel strategy for measuring stiffness in vivo via mechanoscopy (i.e., a system for measuring gut stiffness). Using mechanoscopy, we can quantify gut stress-strain curves and score gut stiffness via the GSI, which is based on the slope of the stress-strain curves. When comparing acute colitis with chronic colitis or when comparing different regions of colon in chronic colitis, we showed that the higher the GSI, the greater the extent of gut fibrosis, the more severe the colon mucosal damage, and the more excessive the infiltrated immune cells. Based on these positive correlations between GSI and IBD severity, GSI and mechanoscopy can be applied in IBD diagnosis, among other tissue abnormalities and aid in the determination of areas of interest or concern. In addition, we demonstrated that the operation of mechanoscopy occurs under physiological loading conditions without inducing any tissue damage.

Using the DSS-induced colitis mouse model, we observed that the stress-strain response significantly diverged towards higher stiffness in the case of chronic colitis compared to healthy control. Further, the magnitude of the strain had to exceed a critical value (e.g., ε=1.5) to identify the divergence. Prior to reaching this strain level, the differentiation of the biomechanical response was not significant between the healthy control and chronic colitis model. This could be explained, for example, that beyond the critical strain, the fibrotic collagen could switch configuration from an undulated state to a straight and tension-loaded state, thus contributing to stiffening at high strain levels. These results may suggest that stiffening of the pathological fibrotic gut can be detected only when it surpasses the critical strain threshold (ε=1.5).

Although no significant difference was detected in the GSI of acute colitis versus healthy tissue, there was a mild increase in the outer diameter of the colon in the acute colitis condition. In chronic colitis, GSI, colon wall thickness, and colon outer diameter were all significantly increased. Thus, two variables could be used to detect colitis: gut outer diameter (geometric variable) and GSI (biomechanical response variable). An increase of only the gut outer diameter suggests inflammation without gut stiffening, whereas an increase of both the outer diameter and GSI suggests more severe inflammation with gut stiffening. Taken together, these findings support mechanoscopy as a promising avenue for the translation application of gut diameter and stiffness measurement in IBD diagnosis.

According to examples described herein, in mechanoscopy, pressure and geometry data are captured and processed to yield stress and strain data, which are used alongside remodeled geometry data in a software program to detect statically significant changes vs. normal response. Furthermore, we have taken the statically significant findings based on the mechanical response and identified their association with disease severity (e.g., using histological analysis of the tissue). In some examples, the mechanoscope system can include predictive capabilities and can incorporate artificial intelligence-based algorithms or other decision-making trees. Using this system and method, we can diagnose soft tissue disease directly based on mechanical response without the need for histology.

Thus, examples of the disclosed technology can provide an improvement over conventional systems and methods for in vivo characterization of soft tissue. The previous description of the disclosed examples is provided to enable any person skilled in the art to make or use the disclosed technology. Various modifications to these examples will be readily apparent to those skilled in the art, and the generic principles defined herein may be applied to other examples without departing from the spirit or scope of the disclosed technology. Thus, the disclosed technology is not intended to be limited to the examples shown herein but is to be accorded the widest scope consistent with the principles and novel features disclosed herein

Unless otherwise specified or limited, the terms “about” and “approximately,” as used herein with respect to a reference value, refer to variations from the reference value of ±15% or less, inclusive of the endpoints of the range. Similarly, the term “substantially,” as used herein with respect to a reference value, refers to variations from the reference value of ±5% or less, inclusive of the endpoints of the range.

Also as used herein, unless otherwise limited or defined, “or” indicates a non-exclusive list of components or operations that can be present in any variety of combinations, rather than an exclusive list of components that can be present only as alternatives to each other. For example, a list of “A, B, or C” indicates options of: A; B; C; A and B; A and C; B and C; and A, B, and C. Correspondingly, the term “or” as used herein is intended to indicate exclusive alternatives only when preceded by terms of exclusivity, such as “either,” “one of,” “only one of,” or “exactly one of.” For example, a list of “one of A, B, or C” indicates options of: A, but not B and C; B, but not A and C; and C, but not A and B. A list preceded by “one or more” (and variations thereon) and including “or” to separate listed elements indicates options of one or more of any or all of the listed elements. For example, the phrases “one or more of A, B, or C” and “at least one of A, B, or C” indicate options of: one or more A; one or more B; one or more C; one or more A and one or more B; one or more B and one or more C; one or more A and one or more C; and one or more of A, one or more of B, and one or more of C. Similarly, a list preceded by “a plurality of” (and variations thereon) and including “or” to separate listed elements indicates options of multiple instances of any or all of the listed elements. For example, the phrases “a plurality of A, B, or C” and “two or more of A, B, or C” indicate options of: A and B; B and C; A and C; and A, B, and C.

In some examples, aspects of the disclosed technology, including computerized implementations of methods according to the disclosed technology, can be implemented as a system, method, apparatus, or article of manufacture using standard programming or engineering techniques to produce software, firmware, hardware, or any combination thereof to control a processor device (e.g., a serial or parallel general purpose or specialized processor chip, a single- or multi-core chip, a microprocessor, a field programmable gate array, any variety of combinations of a control unit, arithmetic logic unit, and processor register, and so on), a computer (e.g., a processor device operatively coupled to a memory), or another electronically operated controller to implement aspects detailed herein. Accordingly, for example, configurations of the disclosed technology can be implemented as a set of instructions, tangibly embodied on a non-transitory computer-readable media, such that a processor device can implement the instructions based upon reading the instructions from the computer-readable media. Some examples of the disclosed technology can include (or utilize) a control device such as an automation device, a special purpose or general purpose computer including various computer hardware, software, firmware, and so on, consistent with the discussion below. As specific examples, a control device can include a processor, a microcontroller, a field-programmable gate array, a programmable logic controller, logic gates etc., and other typical components that are known in the art for implementation of appropriate functionality (e.g., memory, communication systems, power sources, user interfaces and other inputs, etc.). In some examples, a control device can include a centralized hub controller that receives, processes and (re) transmits control signals and other data to and from other distributed control devices (e.g., an engine controller, an implement controller, a drive controller, etc.), including as part of a hub-and-spoke architecture or otherwise.

Certain operations of methods according to the invention, or of systems executing those methods, may be represented schematically in the FIGS. or otherwise discussed herein. Unless otherwise specified or limited, representation in the FIGS. of particular operations in particular spatial order may not necessarily require those operations to be executed in a particular sequence corresponding to the particular spatial order. Correspondingly, certain operations represented in the FIGS., or otherwise disclosed herein, can be executed in different orders than are expressly illustrated or described, as appropriate for particular embodiments of the invention. Further, in some embodiments, certain operations can be executed in parallel, including by dedicated parallel processing devices, or separate computing devices configured to interoperate as part of a large system.

As used herein in the context of computer implementation, unless otherwise specified or limited, the terms “component,” “system,” “module,” “block,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

In some implementations, devices or systems disclosed herein can be utilized, manufactured, installed, etc. using methods embodying aspects of the invention. Correspondingly, any description herein of particular features, capabilities, or intended purposes of a device or system is generally intended to include disclosure of a method of using such devices for the intended purposes, of a method of otherwise implementing such capabilities, of a method of manufacturing relevant components of such a device or system (or the device or system as a whole), and of a method of installing disclosed (or otherwise known) components to support such purposes or capabilities. Similarly, unless otherwise indicated or limited, discussion herein of any method of manufacturing or using for a particular device or system, including installing the device or system, is intended to inherently include disclosure, as embodiments of the invention, of the utilized features and implemented capabilities of such device or system.

Also as used herein, unless otherwise defined or limited, directional terms are used for convenience of reference for discussion of particular figures or examples or to indicate spatial relationships relative to particular other components or context, but are not intended to indicate absolute orientation. For example, references to downward, forward, or other directions, or to top, rear, or other positions (or features) may be used to discuss aspects of a particular example or figure, but do not necessarily require similar orientation or geometry in all installations or configurations.

Also as used herein, unless otherwise limited or defined, “configured to” indicates that a component, system, or module is particularly adapted for the associated functionality. Thus, for example, a ZZ configured to YY is specifically adapted to YY, as opposed to merely being generally capable of doing so.

Although the presently disclosed technology has been described with reference to preferred examples, workers skilled in the art will recognize that changes may be made in form and detail to the disclosed examples without departing from the spirit and scope of the concepts discussed herein.

1. A system for characterizing soft tissue, the system comprising:
a catheter configured to extend into a soft tissue region of an anatomical body;
a pump for actuating inflation or deflation of the catheter;
a pressure transducer in communication with the pump, the pressure transducer configured to sense pressure within the catheter; and
a control system in communication with: (1) the pump to activate the catheter within the soft tissue region of the anatomical body, and (2) the pressure transducer to record a series of pressure measurements from the catheter,
wherein the control system is configured to coordinate pressure data from the catheter with diameter data from a series of diameter measurements of the soft tissue region, and
wherein the control system is further configured to use at least one of the pressure measurements and the diameter measurements to determine a geometric variable or a biomechanical response variable that corresponds to a characteristic of the soft tissue.

2. The system of claim 1, wherein the geometric variable includes the diameter data.

3. The system of claim 1, wherein the biomechanical response variable is a slope from a plot of stress measurements versus strain measurements,
the stress measurements calculated from pressure data and the diameter data, and
the strain measurements calculated from the diameter data.

4. The system of claim 1, further comprising:
a gut diameter module,
wherein the soft tissue region is a gut and the gut diameter module is configured to identify the gut and detect gut edges to calculate diameter data, and
wherein the diameter measurements of the diameter data is the outer diameter of the gut.

5. The system of claim 4, wherein the gut diameter module includes a camera.

6. The system of claim 1, wherein the control system includes a real-time readout of the pressure data and the diameter data.

7. The system of claim 6, wherein the real-time readout of the pressure data and the diameter data occurs at a frequency between 1.4 Hz and 3 Hz.

8. The system of claim 1, wherein the soft tissue region is characterized in vivo.

9. The system of claim 1, wherein the catheter is a water-filled balloon catheter.

10. The system of claim 1, wherein the soft tissue region is a gut including a large intestine and a small intestine.

11. The system of claim 1, wherein the soft tissue region includes one or more of a jejunum, an ileum, or a colon.

12. The system of claim 1, wherein the control system is further configured to calculate a stress versus strain curve from the pressure and diameter data and generate a gut stiffness index from the slope of the stress versus strain curve.

13. A method of obtaining a gut stiffness index for a soft tissue region, the method comprising:
inserting a catheter into an anatomical space having a soft tissue region;
inflating the catheter within the anatomical space;
inflating a soft tissue lumen within the soft tissue region by contacting the soft tissue lumen with the catheter;
recording corresponding pressure and diameter data of the soft tissue lumen;
calculating a stress versus strain curve from the pressure and diameter data of the soft tissue lumen;
calculating the gut stiffness index from the slope of the stress versus strain curve; and
generating a report including the gut stiffness index.

14. The method of claim 13, further comprising:
detecting the presence of chronic colitis if the strain in the stress strain curve is greater than a critical strain.

15. The method of claim 14, wherein the critical strain is 1.5.

16. The method of claim 13, further using at least one of the pressure and the diameter data to determine a geometric variable or a biomechanical response variable that corresponds to a characteristic of the soft tissue and generate a report related to gut pathology.

17. A method of characterizing soft tissue at a soft tissue region within a gut, the method comprising:
collecting pressure measurements from the soft tissue region for a length of time to provide a first series of data points;
collecting diameter measurements of the gut at the soft tissue region for the length of time to provide a second series of data points;
calculating circumferential stress for each data point using the pressure measurements in the first series of data points and the corresponding diameter measurements in the second series of data points;
calculating circumferential strain for each data point using the diameter measurements in the second series of data points; and
outputting one or more of a geometric variable or a biomechanical response variable that characterizes the soft tissue as a report.

18. The method of claim 17, wherein the geometric variable is an outer diameter of the gut at the soft tissue region.

19. The method of claim 17, wherein the biomechanical response is a gut stiffness index calculated from a stress versus strain plot of the circumferential stress and the circumferential strain.

20. The method of claim 19, wherein the gut stiffness index positively correlates to one or more of: a level of gut fibrosis, severity of mucosa damage to the gut, or infiltration of immune cells.

21. The method of claim 19, wherein the gut stiffness index is calculated when the circumferential strain exceeds a critical strain threshold.

22. The method of claim 21, wherein the critical threshold is 1.5.
